(19) United States
(12) Patent Application Publication
(10) Pub. No.: US 2026/0215834 A1
(43) Pub. Date: Jul. 30, 2026

(54) PROSTATE CANCER LESION TARGETING AND VAPOR NEEDLE TIP TRACKING FOR PROSTATE BIOPSY AND VAPOR THERAPY

(71) Applicant: FRANCIS MEDICAL, INC., Maple Grove, MN (US)

(72) Inventors: ROGER N. HASTINGS, Maple Grove, MN (US); MICHAEL HOEY, Shoreview, MN (US); RICHARD C. KRAVIK, Champlin, MN (US); MARK SCHROM, Forest Lake, MN (US)

(21) Appl. No.: 19/145,875

(22) PCT Filed: Dec. 19, 2023
(86) PCT No.: PCT/US2023/084964

§ 371 Date: Jul. 3, 2025

Related U.S. Application Data
(60) Provisional application No. 63/476,083, filed on Dec. 19, 2022.

(51) Int. Cl. A61B 18/04; A61B 17/34; A61B 18/00; A61B 90/00

(52) U.S. Cl. CPC A61B 18/04; A61B 2017/3413; A61B 17/3474; A61B 2018/00547; A61B 2018/00577; A61B 2090/3782

(57) ABSTRACT

A vapor delivery system is provided that may include any of a number of features. One feature of the vapor delivery system is that it can apply condensable vapor energy to tissue, such as a prostrate, to shrink, damage, or denature the prostate. In some embodiments, the vapor delivery system can include safety features including needle tracking and treatment tracking. Biopsy systems for obtaining biopsy tissue samples from the prostate are also provided. Methods for safe and effective treatment of prostate tissues and obtaining prostate biopsy samples are also presented.

### PRIORITY CLAIM

This patent application claims priority to U.S. provisional patent application No. 63/476,083, titled “PROSTATE CANCER LESION TARGETING AND VAPOR NEEDLE TIP TRACKING FOR PROSTATE BIOPSY AND VAPOR THERAPY,” and filed on Dec. 19, 2022, which is herein incorporated by reference in its entirety.

### CROSS REFERENCE TO RELATED APPLICATIONS

This application is related to PCT/US2020/067532, titled “VAPOR THERAPY SYSTEMS AND METHODS,” filed Dec. 30, 2020, and PCT/US2022/020635, titled “VAPOR THERAPY SYSTEMS AND METHODS,” filed Mar. 16, 2022, which are both fully incorporated herein by reference.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD

The present invention relates to devices and related methods for treatment of prostate cancer using a minimally invasive approach.

### BACKGROUND

The human male prostate can be classified into three zones: the peripheral zone, transition zone, and central zone. Peripheral zone (PZ) comprises about 70% of the volume of a male's prostate. This sub-capsular portion of the posterior aspect of the prostate gland surrounds the distal urethra and 70 to 80% of cancers originate in the peripheral zone tissue. The central zone (CZ) surrounds the ejaculatory ducts and contains about 20-25% of the prostate volume. The central zone is often the site of inflammatory processes. The transition zone (TZ) is the site in which benign prostatic hyperplasia (BPH) develops and contains about 5-10% of the volume of glandular elements in a normal prostate, but can constitute up to 80% of such volume in cases of BPH. The transition zone includes two lateral prostate lobes and the periurethral gland region. There exist natural barriers around the transition zone, i.e., the prostatic urethra, the anterior fibromuscular stroma (FS), and a fibrous plane (FP) between the transition zone and peripheral zone. The anterior fibromuscular stroma (FS) or fibromuscular zone is predominantly fibromuscular tissue.

Approximately 70% to 80% of prostate cancers originate in the peripheral zone of the prostate and may be confined to the peripheral zone. In recent years, there has been an increased interest in focal therapy for prostate cancer, treating only regions of tissue in which cancer has been found following biopsies. Prior art focal therapy treatments, such as with RF ablation energy, may not confine the treatment to the peripheral zone tissue or to tissues within the prostate.

Vapor therapy has been successfully used to ablate cancerous prostate tissue. Prostate cancer may present as one or more focal lesions observed on imaging such as magnetic resonance imaging (MRI). These lesions may be identified and located in an MRI that is taken before vapor therapy. Techniques have been developed to fuse MRI images with Trans-rectal ultrasound (TRUS) images, for example to help guide prostate biopsies to the location of lesions identified on MRI. While image fusion is useful, it is time consuming and must account for extensions of prostate tissue when the TRUS probe is inserted into the rectum and when the vapor delivery shaft is inserted into the urethra.

Improved prostate vapor delivery and biopsy systems and methods are desired.

### SUMMARY

A method is provided, comprising obtaining a first medical image of a prostate including target tissue region and an anatomical landmark; measuring a separation distance and clock angle between the target tissue region and the anatomical landmark; imaging the anatomical landmark under real-time ultrasound imaging; advancing a needle position of a vapor delivery device transurethrally to the anatomical landmark under the real-time ultrasound imaging; further advancing and rotating the needle position of the vapor delivery device transurethrally by the separation distance and clock angle; and deploying a needle of the vapor delivery device from the needle position through the urethra, into the prostate, and into the target tissue region.

In one aspect, the target tissue region comprises prostate cancer tissue.

In some aspects, the anatomical landmark is selected from the group consisting of a base of the prostate, an apex of the prostate, a bladder neck, a verumontanum, and a location where ejaculatory ducts meet the urethra.

In some aspects, the real-time ultrasound imaging comprises a trans-rectal ultrasound imaging (TRUS) probe.

In some aspects, further advancing the needle position further includes advancing the TRUS probe in step with the needle position of the vapor delivery device.

In other aspects, deploying the needle further comprises deploying the needle generally transverse to a shaft of the vapor delivery device.

In some aspects, deploying the needle further comprises deploying the needle along an arc.

In some aspects, the method includes displaying an animation of the arc of the needle on the real-time ultrasound image, translating and/or rotating the delivery device until the animated needle tip intersects an animation of targeted cancerous tissue, then deploying the needle into the target tissue.

In one aspect, the method includes tracking a position of the needle during the deployment.

In some aspects, the method includes displaying the tracked position on the real-time ultrasound imaging.

A medical device gps tracking system is provided, comprising: a trans-rectal ultrasound imaging system (TRUS) probe configured to obtain one or more ultrasound images of a target tissue in an imaging plane; a plurality of sensors disposed on or within the TRUS probe, the plurality of sensors being configured to receive signals from one or more transmitters positioned on a medical device and within the imaging field of view; and control electronics configured to determine a position of the one or more transmitters from the received signals and communicate position data of the medical device for real time display on the one or more ultrasound images.

In some aspects, the plurality of sensors are disposed on a sleeve configured to be placed over the TRUS probe.

In one aspect, the TRUS probe comprises a transverse transducer array, wherein the sleeve has a window or opening corresponding to the transverse transducer array.

In some aspects, the plurality of sensors on or within the TRUS probe comprise induction sensor coils.

In other aspects, the induction sensor coils comprise six induction sensor coils.

In some aspects, the induction coils are wound on faces of a non-metal sensor cube.

In one aspect, the induction coils are positioned on a distal tip of the TRUS probe.

In some aspects, the induction coils are positioned distal and proximal to a sagittal array of the TRUS probe.

In other aspects, the control system is configured to register coordinates of the TRUS probe to coordinates of the plurality of sensors.

A medical system is provided, comprising: a therapy device configured for transurethral access to a patient's prostate, the therapy device having a deployable needle configured to be actuated from a delivery configuration to a deployed configuration and one or more transmitters disposed thereon; a trans-rectal ultrasound imaging system (TRUS) probe; a plurality of sensors disposed on or within the TRUS probe, the plurality of sensors being configured to receive signals from the one or more transmitters; control electronics configured to determine a position the one or more transmitters from the received signals and communicate position data to the TRUS probe; and a display configured to display real-time images of the prostate from the TRUS probe overlaid with the position of the one or more transmitters.

In some aspects, the plurality of sensors are disposed on a sleeve configured to be placed over the TRUS probe.

In another aspect, the TRUS probe comprises transverse and/or sagittal transducer arrays, wherein the sleeve has a window or opening corresponding to the transverse and/or sagittal transducer arrays.

In some aspects, the plurality of sensors on or within the TRUS probe comprise induction sensor coils.

In another aspect, the induction sensor coils comprise six induction sensor coils.

In some aspects, the induction coils are wound on faces of a non-metal sensor cube.

In other aspects, the induction coils are positioned on a distal tip of the TRUS probe.

In some aspects, the induction coils are positioned distal and proximal to a sagittal array of the TRUS probe.

In another aspect, the control system is configured to automatically register coordinates of the TRUS probe to coordinates of the plurality of sensors.

A biopsy system is provided, comprising: a shaft configured for transurethral access to a prostate of a patient; a needle disposed in the shaft, the needle having a delivery configuration in which it is fully positioned in the shaft and a deployed configuration in which it is advanced generally transverse to the shaft; and a tissue sample collector within the needle and configured to be advanced beyond a distal tip of the needle; wherein the biopsy system is configured to obtain a tissue sample by deploying the needle and tissue sample collector to a position proximal to the tissue sample, advancing the tissue sample collector to a position distal to the tissue sample, and then advancing the needle over the tissue sample to capture the tissue sample inside the needle and tissue sample collector.

In some aspects, the tissue sample collector includes a trough configured to collect the tissue sample.

In some aspects, the system includes one or more electrodes positioned on a tip of the tissue sample collector.

In other aspects, the one or more electrodes are configured to cauterize tissue.

In some aspects, the one or more electrodes are configured to measure an impedance of the prostate.

In another aspect, the needle comprises a distal cutting edge, wherein the distal cutting edge is configured to slice the tissue sample when the needle is advanced to capture the tissue sample inside the tissue sample collector.

In some aspects, the tissue sample collector is removable from the needle.

In other aspects, the needle and the tissue sample collector are controlled with one or more linear drive motors.

A biopsy system is provided, comprising: a shaft configured for transurethral access to a prostate of a patient; a needle disposed in the shaft, the needle having a delivery configuration in which it is fully positioned in the shaft and a deployed configuration in which it is advanced generally transverse to the shaft; and a tissue sample collector within the needle configured to be deployed into prostate tissue with the needle; wherein the biopsy system is configured to obtain a tissue sample by deploying the needle and tissue sample collector to a position distal to the tissue sample, retracting the needle to a position proximal to the tissue sample, and then advancing the needle over the tissue sample collector to capture the tissue sample inside the tissue sample collector.

A method of obtaining a prostate tissue sample is provided, comprising: inserting a shaft of a biopsy device into a patient's urethra; advancing and rotating the shaft transurethrally to engage tissue selected for biopsy; deploying a needle and sample collector of the biopsy device into the prostate from the urethra until a distal end of the needle is proximal to the prostate tissue sample; advancing a tissue sample collector from the needle to a location that is distal to the tissue sample; holding the tissue sample collector in place while advancing the needle to capture the prostate tissue sample within the needle.

A method of obtaining a prostate tissue sample is provided, comprising: inserting a shaft of a biopsy device into a patient's urethra; advancing and rotating the shaft transurethrally to engage tissue selected for biopsy; deploying a needle and sample collector of the biopsy device into the prostate from the urethra until a distal end of the sample collector is distal to the desired prostate tissue sample; retracting the needle to a location that is proximal to the desired tissue sample; holding the tissue sample collector in place while advancing the needle to capture the prostate tissue sample within the sample collector.

In some aspects, the method includes retracting the tissue sample collector proximally through the biopsy device.

In other aspects, the method includes retracting the needle and the tissue sample collector from the patient.

A biopsy system is provided, comprising: a shaft configured for transurethral access to a prostate of a patient; a needle disposed in the shaft, the needle having a delivery configuration in which it is fully positioned in the shaft and a deployed configuration in which it is advanced generally transverse to the shaft; and an auger configured to be advanced from the needle to capture a tissue sample into the auger.

A method of obtaining a prostate tissue sample is provided, comprising: inserting a shaft of a biopsy device into a patient's urethra; advancing the shaft transurethrally to the prostate; deploying a needle of the biopsy device into the prostate from the urethra until a distal end of the needle is proximal to the prostate tissue sample; and advancing an auger from the needle through the tissue sample to capture the prostate tissue sample within the auger.

### BRIEF DESCRIPTION OF THE DRAWINGS

To better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIGS. 1A-1B show anatomical models of the prostate.

FIG. 2 shows a vapor therapy system including a vapor delivery device and a TRUS probe.

FIGS. 3-7 show TRUS and or MRI images of the prostate.

FIG. 8 shows one embodiment of a vapor delivery device including a vapor delivery needle that deploys outwards from the shaft of the device along an arc.

FIG. 9 shows one embodiment of a vapor therapy system including a vapor delivery device and a TRUS probe.

FIGS. 10A-10C show a TRUS probe having a GPS sleeve.

FIGS. 11A-11B and FIG. 12 show a vapor delivery device with a GPS or tracking sensor.

FIG. 13 shows a schematic of a needle tracking system.

FIGS. 14-17 show embodiments of a needle biopsy system.

FIGS. 18A-18G shows another embodiment of a needle biopsy system and methods of use.

FIGS. 19A-19C show another embodiment of a needle biopsy system.

FIG. 20 shows a vapor delivery sheath inserted into the needle following biopsy to cauterize the needle exit hole in the wall of the urethra as the needle is retracted into the delivery device shaft.

### DETAILED DESCRIPTION OF THE INVENTION

Systems and methods are provided herein for treating cancer of the prostate. The systems and methods herein are configured to introduce a heated vapor interstitially into the interior of a prostate to controllably ablate prostate tissue. The systems and method herein are configured to cause localized ablation of prostate tissue without damaging the prostatic urethra and without damaging tissue outside of the prostate gland.

Additionally, some systems and methods provided herein are directed to the treatment of prostate cancer, and more particularly for ablating peripheral zone prostate tissue without ablating central or transitional zone prostate tissue.

Systems provided herein can include a vapor needle that delivers vapor media transurethrally into the prostate. The system can include a vapor generator configured to produce the vapor from a fluid media in real-time during a procedure.

In some embodiments, a prostate treatment device can include an introducer shaft sized and configured for transurethral access into a patient, a vapor generator configured to generate a condensable vapor, a vapor delivery needle in communication with the vapor generator and slidably disposed within the introducer shaft, and an actuator configured to move the vapor delivery needle between a retracted position inside the introducer shaft and an extended position at least partially outside of the introducer shaft, and to advance or retract the needle continuously or in steps to tissues at any location between the prostatic urethra and prostate capsule.

This disclosure is directed to safe and effective delivery of vapor to ablate tissue. A vapor delivery device can include a shaft configured for transurethral access to a patient's prostate, a vapor generator, and a vapor delivery needle that can include one or more vapor delivery ports. In one embodiment vapor is delivered through the port(s) of the vapor delivery needle to ablate cancerous or precancerous tissue. In a preferred embodiment, the vapor delivery needle is configured to puncture the prostatic urethra and advance to one or more sites within the prostate where vapor is delivered. Multiple puncture sites can be spaced apart to provide overlapping zones of tissue ablation in the prostate, without being close enough together to allow vapor delivered at a site to exit through the entry holes of the previous puncture sites.

More specifically, this disclosure is directed to navigation and tracking of a vapor delivery device, including tracking and/or visualization of the vapor delivery needle including the needle arc or deployment, into and throughout the prostate to ablate cancerous tissue and avoid penetrating the prostate capsule. Vapor is delivered to sites that are surrounded by tissue that has been targeted for ablation.

This disclosure provides systems and methods to quantify translation of lesion coordinates from pre-operative MRI images to images obtained with the TRUS or other ultrasound imaging systems. The methods provide guidance for vapor delivery device placement that will bring vapor delivery holes, located near a needle tip of the vapor delivery device, to the site of a lesion.

One targeting method of this disclosure relies on the location of a cancerous lesion in the prostate identified on pre-operative MRI image(s). The separation between the apex of the prostate and the transverse plane that contains the cancerous lesion can be measured on the MRI image. The vapor delivery device can then be advanced from the apex by the measured separation, to place the needle exit near the transverse plane of the lesion. An operator of the device can estimate the clock angle of the lesion and its separation from the urethra. The vapor delivery needle can be deployed at the estimated clock angle and advanced until the estimated position of the needle is at the estimated centroid of the lesion.

This targeting method relies on knowledge of the arc of the vapor delivery needle as it is deployed away from the delivery device shaft as a function of the known needle deployment length. The needle arc is known and quantified when the needle is delivered into air or homogenous soft substances. Changes in needle arc due to tissue inhomogeneities or accidental movement of the delivery device shaft may result in faulty lesion targeting.

A real-time measurement of the location of the vapor needle tip is provided herein to ensure that the needle tracks to the lesion centroid for vapor delivery. This disclosure provides systems and methods for tracking the trajectory of the needle tip and displaying its location on an ultrasound or MRI image of the prostate. This disclosure provides sense coils that can be integrated onto or into the TRUS probe so that needle tip location is automatically given relative to the real time TRUS image. In some examples, no coils, cables or other hardware are required outside the patient. Multiple TRUS-mounted sensors can be configured to simultaneously detect an AC magnetic field generated by a coil of fine wire wound near the tip of the vapor delivery needle. Sensor outputs can then be converted to needle tip location and orientation in software and presented to the user. Real-time knowledge of the needle location and orientation before and after needle deployment from the delivery device shaft facilitates the lesion targeting methods and provides real-time measurements of the path of the needle tip as it advances from the urethra to the lesion.

The lesion targeting methods of this disclosure can also be applied to a novel transurethral prostate tissue biopsy system that is integrated with or separate from a vapor delivery system. Conventional transrectal or trans-perineum biopsy needles cross body tissues before entering the prostate, enabling the potential transfer of harmful bacteria into the prostate as the needle is deployed, especially in the transrectal approach. When the needle is extracted, prostate cancer cells may be transferred to body tissue surrounding the needle, with the potential for prostate cancer metastasis. Patients experience bleeding from the needle insertion holes that may last for weeks following the procedure. In the transurethral biopsy system of this disclosure, the biopsy needle is delivered to tissue through the wall of the urethra using the same or similar system and methods as the vapor therapy system. The needle passes from the sterile external space of urethra to the prostate without passing through intervening tissue. A short burst of steam may be applied as the needle exits the urethral wall, thereby sterilizing and cauterizing the exit hole to prevent bleeding and spreading of cancer cells.

This disclosure provides systems and methods for navigating percutaneous devices to the site of focal prostate cancer lesions that have been identified on MRI images of the prostate. In some embodiments vapor may be delivered to the site of the lesion while the patient is in the MRI machine. Real-time MRI guidance has the advantage of identifying both the lesion and vapor delivery needle tip in real time. In addition, MRI can provide a real time, color-coded map of temperature in the prostate tissue, providing a visual and quantitative image of tissue ablation as it occurs. In general, the vapor delivery device shaft, the needle and needle tip, and the cancerous lesion are all visible during the MRI procedure. MRI enhancing markers may be placed at critical locations on the vapor device. The delivery device must be constructed from MRI-compatible materials. The methods of lesion targeting discussed below may be applied to real time MRI guidance through the urethra.

Ultrasound guidance to a lesion identified on a preoperative MRI image requires the additional steps of translating the lesion location from the MRI to the ultrasound image. In some embodiments of ultrasound guided lesion targeting the vapor delivery device shaft is advanced from a feature that is visible on both the MRI and ultrasound images (for example the apex or base of the prostate or the verumontanum) to the transverse plane of the lesion by a distance that is measured on the MRI image. The distance traversed by the delivery device shaft may be measured by a sensor located on the delivery device shaft or on the needle tip transmit coil retracted into the shaft. In other examples, especially when there are no position sensors on the delivery device, the distance is measured as the location of the transverse plane of the ultrasound image as it is advanced from a prostate feature to the plane containing lesion identified on MRI, then advancing the delivery device shaft until its tip is visible on the TRUS image.

Once the delivery device shaft tip is near the plane of the lesion, the shaft may be rotated to an angle that places the plane of the deployed needle into the plane of the lesion. In a qualitative approach the operator estimates the required rotation angle simply be observing the lesion on the MRI image and estimating the clock angle of the lesion relative to the urethra. If the urethra is not visible on the MRI image, its location is estimated by observing it on the ultrasound image.

More quantitative approaches to lesion targeting establish scale factors that translate Cartesian (x, y, z) coordinates of the MRI images the ultrasound images. Distances may be measured on either image, then translated to the other through the measured scale factors. Rotation angles are computed from measured coordinates on the MRI image and translated to the TRUS image. Targeting angles are determined by measuring and computing distances along the x and y axes between the delivery device shaft in the urethra and the lesion centroid. The vapor delivery needle is deployed at a location in the urethra and an angle that is determined by the known arc of the needle and known total length of needle that has been deployed. Animations of the tumor centroid, the delivery device shaft and deployed needle may be superimposed on the real time TRUS image so the operator can translate and rotate the delivery device with the needle retracted to determine the angle and needle deployed distance at which the vapor delivery holes near the needle tip will intersect the tumor centroid.

In some embodiments the pre-operative MRI image can be fused to the real time ultrasound image. The target lesion then is visible on the fused image and the delivery device tip may be brought to the plane of the lesion via visual guidance. Qualitative or quantitative methods may then be used to determine the location and angle of the shaft for needle deployment.

The lesion targeting methods of this disclosure are applied to a novel transurethral prostate tissue biopsy system that may be integrated with the vapor delivery system. Conventional transrectal or trans-perineum biopsy needles cross body tissues before entering the prostate, enabling the potential transfer of harmful bacteria into the prostate as the needle is deployed, especially in the transrectal approach. When the needle is extracted, prostate cancer cells may be transferred to body tissue surrounding the needle, with the potential for prostate cancer metastasis. Patients experience bleeding from the needle insertion holes that may last for weeks following the procedure. In the transurethral biopsy system of this disclosure, the biopsy needle is delivered to tissue through the wall of the urethra using the same or similar techniques and methods as the vapor therapy system. The needle passes from the sterile external space of urethra to the prostate without passing through intervening tissue. A short burst of steam may be applied as the needle exits the urethral wall, or a short burst of current applied between needle tip electrodes (for example bio-impedance electrodes), thereby sterilizing, and cauterizing the exit hole to prevent bleeding. At some point following histo-pathological examination of the tissue samples, the vapor delivery device needle can then be delivered to the same sites where tissues were collected using the same system. Post vapor therapy samples may be collected from these same sites using the same system to determine therapy outcomes.

### Prostate Anatomy and Landmarks

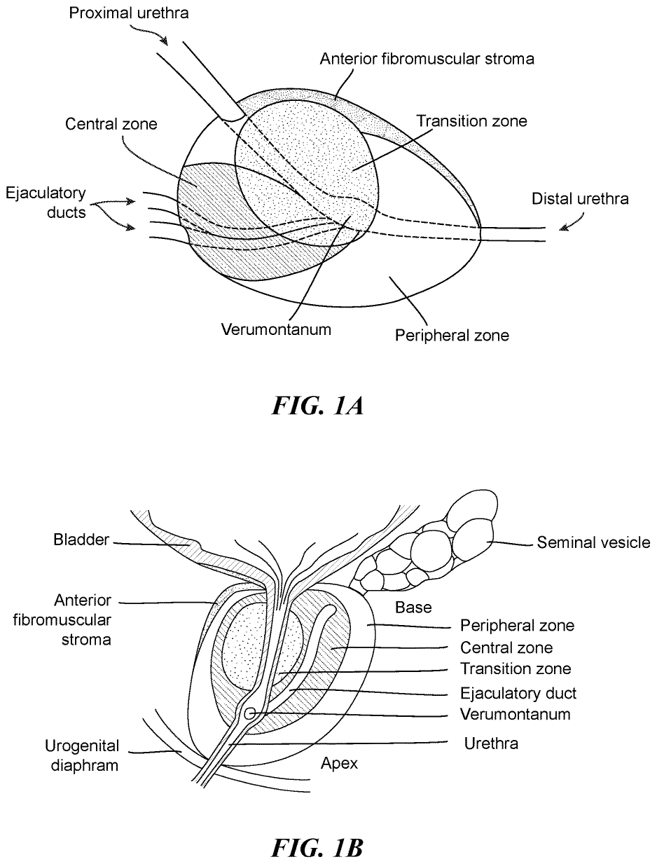

Sagittal and coronal views of the human prostate are shown in FIGS. 1A-1B with anatomical features identified. Zones of the prostate are identified. Most cancers originate in the peripheral zones. Landmarks used to cross reference MRI and TRUS images include the base (or bladder neck) and apex of the prostate, and the verumontanum or location where the ejaculatory ducts meet the urethra.

### Delivery Device and TRUS Probe Locations

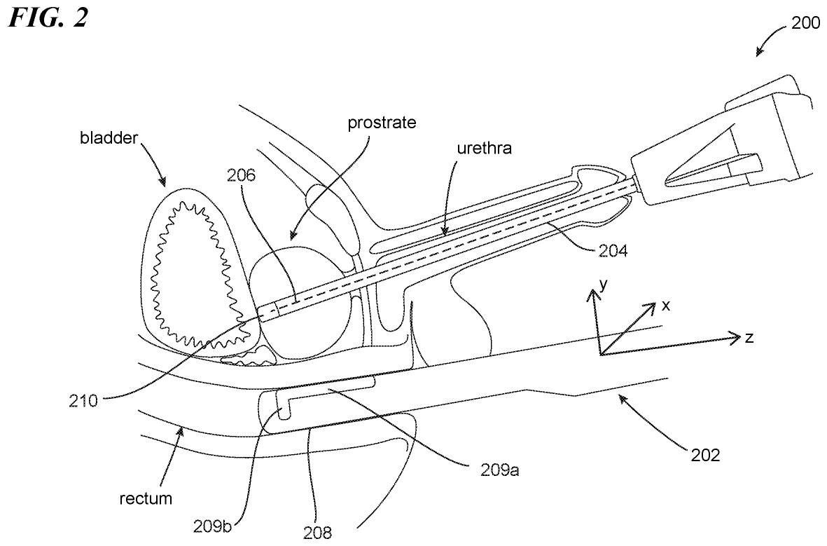

A vapor delivery device according to the present disclosure generally has a number of features to facilitate transurethral delivery of vapor to the prostate. For example, referring to FIG. 2, a vapor delivery device 200 may include an elongate shaft 204, a translatable vapor delivery needle 206 disposed within the shaft and deployable from the shaft (e.g., into prostate tissue), and a vapor generator disposed in the device (not shown) and fluidly coupled to the vapor delivery needle. Additionally, the systems and methods disclosed herein can also include a trans-rectal ultrasound (TRUS) probe 202 including an array 208 configured for providing real-time ultrasound images of the prostate in two or more planes. As shown, the array 208 includes a sagittal array 209a and a transverse array 209b.

In FIG. 2, the vapor delivery device 200 and TRUS probe 202 are shown inserted into the urethra and rectum of a subject, respectively. Both the tip 210 of the vapor delivery device shaft 204 and an imaging plane of the TRUS array 208 are at the base of the prostate as shown. Cartesian coordinates used for lesion targeting are shown. As the devices are moved along the Z axis during a procedure (e.g., into or out of the urethra/rectum), the delivery device shaft tip is a key feature identified on the TRUS probe transverse and sagittal images. In some aspects, it can be important to ensure that the elongate shaft 204 and the TRUS probe 202 are parallel to each other for quantitative lesion targeting. The sagittal array 209b provides a cross sectional view of the prostate in the plane shown. In some aspects, the system can include stabilizer arms or other features configured to lock down the vapor delivery device and TRUS probe during the procedure. The elongate shaft of the vapor delivery device may be rotated and the TRUS probe may be translated or rotated while their central locations are locked down relative to the patient.

### Qualitative Lesion Targeting

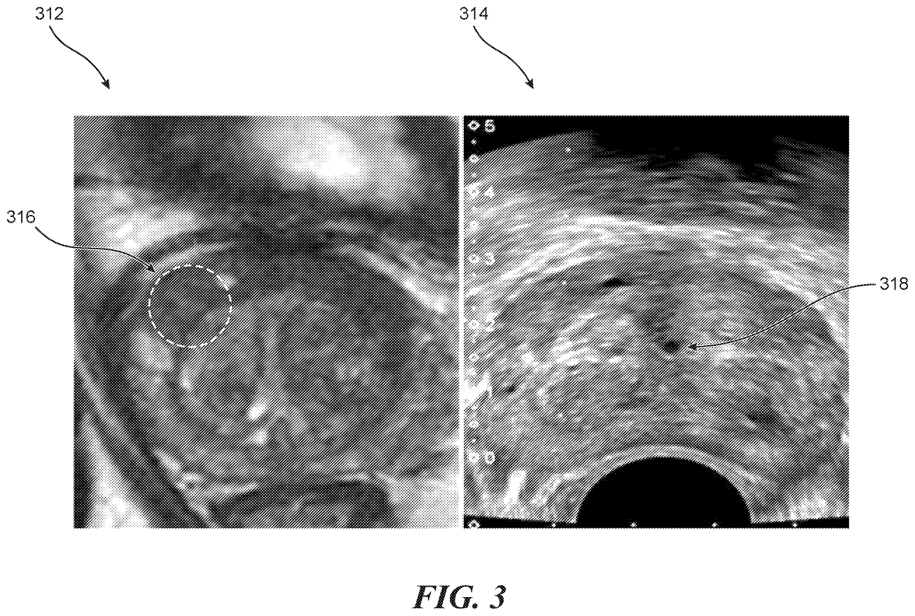

A method of positioning a vapor delivery device at a lesion is now described. A transverse MRI image 312 of a human prostate is shown on the left side of FIG. 3, with a transverse TRUS image 314 (e.g., from the TRUS probe described above) is shown on the right side. A lesion 316 is identified in the MRI image 312. Additional transverse MRI images can be used to identify an anatomical landmark in the prostate (e.g., the apex of the prostate). It should be understood that if a first transverse MRI image (e.g., image 312) shows the lesion and a second transverse MRI image that is spaced axially from the first transverse image shows the anatomic landmark, then the lesion and the anatomical landmark are axially spaced from another. The separation between the MRI plane or image containing the lesion and the MRI plane containing that the anatomical landmark 318, that also appears on either the TRUS image or on the delivery device cystoscope image, is measured. For example, the axial distance between transverse MRI images can be calculated or determined, such as with the MRI system. Generally, a feature or landmark that is closest to the plane of the lesion can be chosen. In an example, the distance between the transverse MRI planes or images containing the lesion and the apex of the prostate can be measured on the MRI image(s) or with the MRI system software.

Once the distance between the lesion and the anatomical landmark is known in the axial direction, the delivery device shaft tip can be inserted into the patient's urethra and the transverse plane of the TRUS probe can be transrectally positioned at the anatomical landmark (e.g., positioned at the apex of the prostate). The delivery device can then be advanced within the urethra by the separation or measured distance between the lesion and the anatomical landmark to bring it to the transverse plane of the lesion. It is noted that the TRUS probe is advanced along with the delivery device to maintain the delivery device shaft tip in the transverse imaging plane of the TRUS probe.

At this point in the lesion targeting technique, the delivery device may be advanced by a distance equal to the known/measured separation between the shaft tip and the vapor needle exit hole(s). The vapor delivery device can be further translated and rotated to an angle that will bring the needle after deployment into the plane of the lesion centroid. The needle deployed length can be displayed on the system console and may be used to estimate the location of the vapor delivery holes, which may be just proximal of the delivery device tip. The needle is deployed and advanced by a length that will bring the needle tip into the lesion. In some embodiments, animations on the TRUS display screen may aid in targeting. For example, an animation of the delivery device shaft with an estimated arc of the needle may be displayed on a display of the system along with or overlaid on the TRUS image. The approximate lesion centroid may be marked on the TRUS image, and the needle may be translated and rotated until the animated needle arc crosses this mark. When vapor is delivered, the steam appears brightly on the TRUS image, showing the location of the vapor proximate the needle tip.

### Quantitative Lesion Targeting

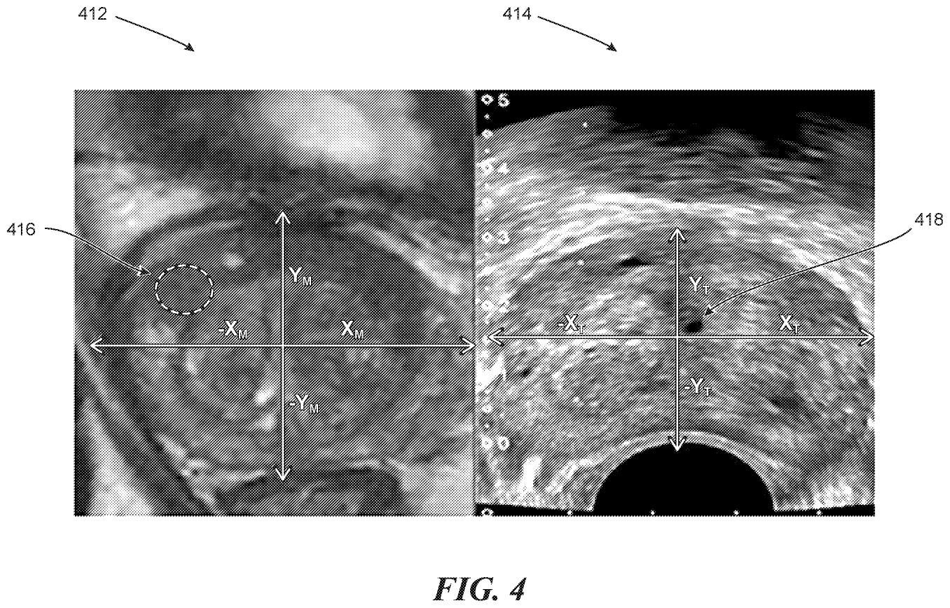

Referring to FIG. 4, a more quantitative approach to lesion targeting begins with a mathematical scaling of MRI coordinates to TRUS coordinates to account for scale differences due to the presence of the TRUS probe and delivery device shaft in the anus and urethra respectively, and any changes in the prostate between the time of the pre-operative MRI and vapor therapy procedures. In FIG. 4, the coordinates in the X/Y axis for the MRI image 412 are shown as +/−X_{M} and Y_{M}, and the coordinates in the X/Y axis for the TRUS image 414 are shown as +/−X_{T} and Y_{T}.

A simple scaling has:

$${X}_{T} = a {X}_{M} \qquad ( 1 )$$

$${Y}_{T} = b {Y}_{M}$$

$${Z}_{T} = c {Z}_{M}$$

$$where T = TRUS and M = MRI$$

In one embodiment, the procedure begins with the tip of the delivery device shaft and the plane of the transverse TRUS image at the base of the prostate (as previously described with respect to FIG. 2). The delivery device can be rotated so that the vapor delivery needle, once deployed, will lie in the sagittal plane of the TRUS image. The delivery device can be tilted until it is parallel to the TRUS probe. The separation between an anatomical landmark such as the base and apex of the prostate is measured on both the TRUS, “D_{T}”, and MRI, “D_{M}”, sagittal images, and the Z axis scale factor in Eq.(1) is calculated as:

$$c = {D}_{T} / {D}_{M} \qquad ( 2 )$$

The transverse plane of the lesion 416 is identified on the MR image. The Z-axis distance between the anatomical landmark 418 (e.g., the base of the prostate) and the plane of the lesion is measured on the MR image. The TRUS Z-axis distance between the base of the prostate and the plane of the lesion is computed from Eqs. (1) and (2), Z_{T}=c Z_{M}. Both the tip of the delivery device shaft and the transverse plane of the TRUS are moved the distance Z_{T} to the plane of the lesion. If the plane of the lesion is closer to another landmark, for example the verumontanum or the apex of the prostate, the distance Z_{T} may be measured from the closer landmark to the plane of the lesion.

Images of the prostate at the plane of the lesion on the MR image and at the computed plane of the lesion on the TRUS image are shown side by side in FIG. 4. Scale factors “a” and “b” in Eq.(1) are determined by dividing the image in half both horizontally and vertically, and measuring the distance between arrow tips on both images. The X and Y coordinates shown are then half of the distance between arrow tips, and the scale factors are: a=X_{T}/X_{M} and b=Y_{T}/Y_{M}.

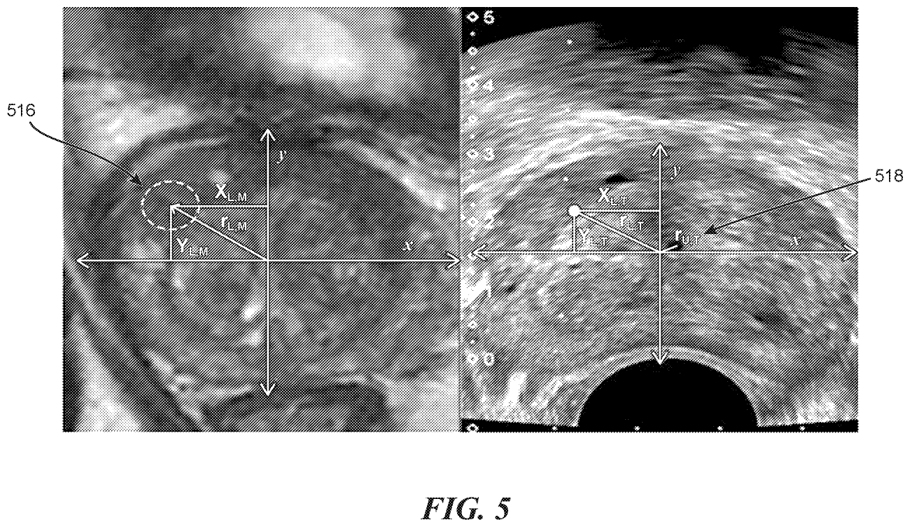

Next, referring to FIG. 5, the coordinates of the lesion centroid 516 relative to the image center are measured on the MRI image and scaled to the TRUS image. The goal is to compute the coordinates of the lesion 516 centroid relative to the delivery device located in the urethra at 518. The MRI and TRUS coordinates of the lesion and the vector location of the urethra on the TRUS image are shown in FIG. 5. The TRUS coordinates are derived from the MRI coordinates using the scale factors “a” and “b” computed above: X_{L,T}=aX_{L,M} Y_{L,T}=bY_{L,M}.

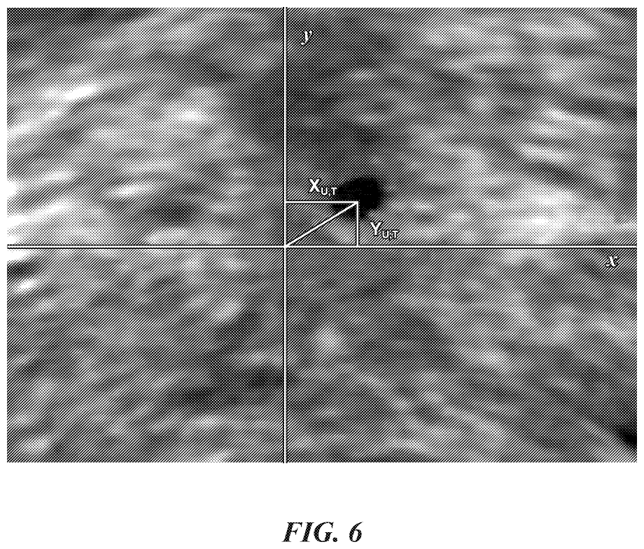

The coordinates of the urethra relative to the TRUS coordinate origin (X_{U,T}, Y_{U,T}) are shown in FIG. 6. They are measured on the TRUS image in the transverse plane that contains the lesion.

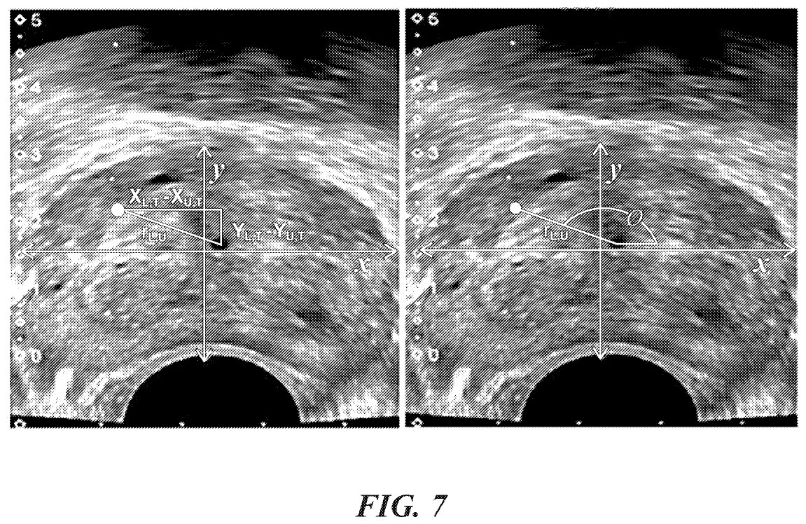

Next, the coordinates of the lesion centroid are computed relative to the tip of the delivery device shaft, located in the urethra. The coordinates are shown in FIG. 7, and both Cartesian and polar coordinates are computed below:

$${X}_{L , U} = {X}_{L , T} - {X}_{U , T} \qquad ( 3 )$$

$${Y}_{L , U} = {Y}_{L , T} - {Y}_{U , T}$$

$${r}_{L , U} = SQRT ( {X}_{L , U}^{2} + {Y}_{L , U}^{2} )$$

φ=polar angle of the lesion relative to the urethra
1^{st} quadrant: φ=(180/π) ATAN(Y_{L,U}/X_{L,U}), (X_{L,U} and Y_{L,U} positive)
2^{nd} quadrant: φ=180°+(180/π) ATAN(Y_{L,U}/X_{L,U}), (X_{L,U} negative and Y_{L,U} positive)
3^{rd} quadrant: φ=180°+(180/π) ATAN(Y_{L,U}/X_{L,U}), (X_{L,U} negative and Y_{L,U} negative)
4^{th} quadrant: φ=360°+(180/π) ATAN(Y_{L,U}/X_{L,U}), (X_{L,U} positive and Y_{L,U} negative)

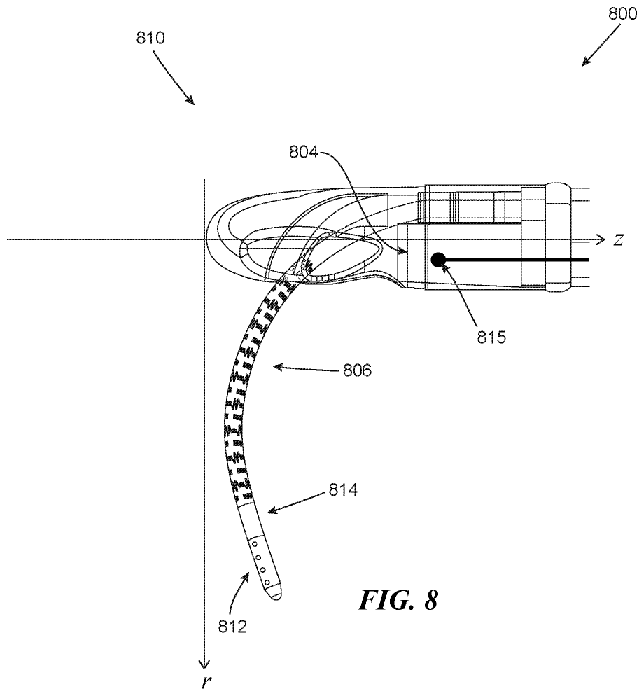

FIG. 8 shows a vapor delivery device 800 including showing aa vapor delivery needle 806 extending outwards from a distal tip 810 of the device shaft along an arc. As shown, the vapor delivery needle can include one or more vapor delivery ports 812 configured to deliver vapor or steam into a targeted tissue. In this figure, the Z-axis is parallel to the delivery device (e.g., along the longitudinal axis of the device shaft). Additionally, the vapor delivery needle 806 and/or shaft 804 can include one or more sensors 814/815, such as GPS sensors, rotational sensors, accelerometers, transmitters, needle guidance system (NGS) sensors, needle position or deployment tracking sensors, or coils. The distance between the tip 810 of the delivery device shaft and a given point or location on the needle is defined by a distance “r” from the centerline of the delivery device shaft, in the plane of the arc. The tip of the delivery device can be moved axially by a distance Z (e.g., within the urethra) to ensure that the tip of the needle will intersect the lesion centroid.

During a procedure, the delivery device may be rotated to the polar angle of the lesion, φ, computed above (the angle φ may be measured with a protractor mounted on the delivery device or by an angle sensor). This ensures that the plane of the needle after deployment will lie in the plane of the lesion. It may be assumed that the needle will deploy in a pre-measured arc observed in the lab when the needle is deployed into air or phantom tissue. In some examples, the needle arc can be defined by an arc equation where z=0.0006r³+0.0032r²−0.3497r+7.2875, where “r” is the polar distance between the delivery device in the urethra and the lesion centroid given in Eq.(3) above. The delivery device can be moved axially by a distance Z computed in FIG. 8 from the arc equation. In some aspects, a display of the system can instruct or guide a user to move the device by the computed distance Z. This ensures that the needle tip and adjacent vapor delivery holes will pass through the centroid of the lesion located at polar coordinates (r,φ).

After following the steps above during a procedure, the needle is now deployed at angle φ into the plane of the lesion. The needle deployed length, “s”, can be measured by sensors (e.g., one or more sensors 814) within the needle deployment mechanism, shaft, or on the needle itself, and be displayed to the user. The deployed length that corresponds to the distance “r” to the lesion is given by:

$$s = 0.0082 {r}^{2} + 0.8001 r - 2.3181 \qquad ( 5 )$$

The deployed needle length is computed for the radial distance to the lesion computed in Eq.(3). The needle is advanced until the measured deployed length is within one mm of the length computed from Eq.(5). Vapor can then be delivered to at this computed location of the lesion centroid.

### Sensor Guided Needle Targeting

Distances in the TRUS images may be measured on the TRUS system, assuming the tip of the delivery device shaft (or other aspect of the system to be measured) is in focus on the ultrasound images. In some embodiments, an electromagnetic or other tracking sensor 815 may be placed on the delivery device shaft to make shaft coordinate measurements. In the targeting methods discussed above, the location of the needle tip is computed from an arc equation that is assumed to be accurate in all circumstances. In a preferred embodiment of this disclosure, a tracking device or electromagnetic field transmitter 814 is placed on or adjacent to the tip of the vapor delivery needle in FIG. 8. The location of the needle tip is thereby known throughout the procedure, most importantly when it deviates from the computed arc.

This disclosure provides a determination of the location and orientation of a current carrying coil or sensor (e.g., transmitter or sensor 814 in FIG. 8) near the tip of a vapor delivery device needle within the prostate gland. The sensor or transmitter 814 can be configured to monitor a needle track or arc of a needle, which can be displayed in real-time ultrasound and/or MRI images of the prostate during a procedure. In a preferred embodiment, the needle coil 814 is a transmitter comprising a multi-turn coil of fine wire, through which a constant amplitude sine wave current can be applied at a preferred frequency. The magnetic field generated by the needle tip coil 814 can be sensed by an array of magnetic sensors in proximity to the transmitter 814. For example, a sensing array may be positioned on or within a trans-rectal ultrasound (TRUS) imaging probe located within the rectum of the patient. In an alternate embodiment 814 comprises a magnetic field sensor that receives electromagnetic fields from an array of nearby transmitters, located for example proximate a TRUS probe.

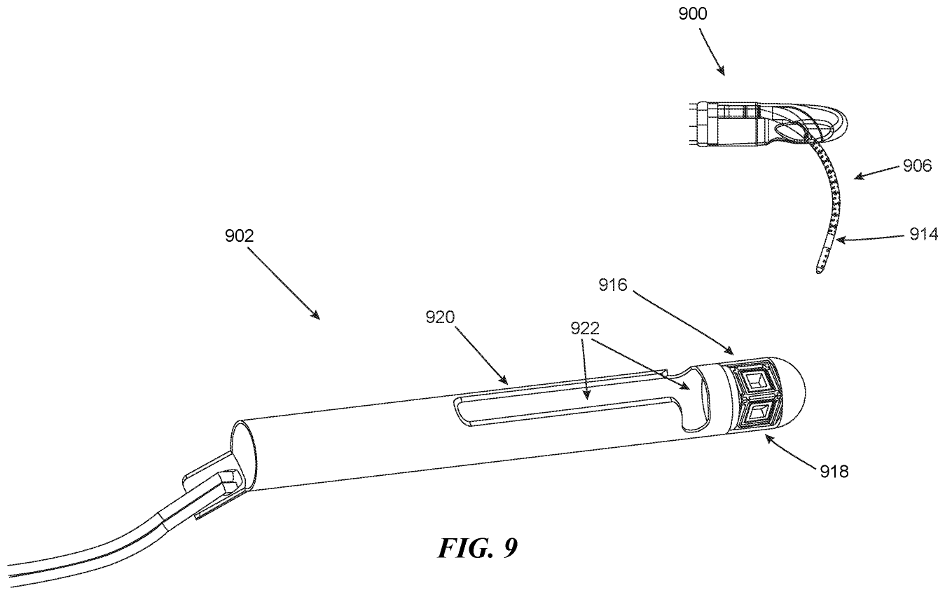

Referring to FIG. 9, a system is shown that includes a vapor delivery device 900 and a TRUS probe 902. As described above, the vapor delivery device can include one or more transmitters or sensors 914 on the vapor delivery needle 906. Additionally, the TRUS probe 902 can include a plurality of induction sensor coils or transmitters 916. In one embodiment, the sensor coils comprise a set of six induction sensor coils 916 that are wound on the faces of a non-metal sensor cube 918 that is integrated into the TRUS probe 902, such as in the distal tip of the TRUS probe, as shown in FIG. 9. The transmitter 914 on the vapor needle can be sensed by the induction sensor coils 916 of the TRUS probe. Voltages sensed in the sensor coils can be inverted in a statistical algorithm to determine the three cartesian coordinates and two orientation angles of the needle tip transmitter 914. Since the centroid of the coils is rigidly fixed relative to the TRUS probe transducers, the location of the needle tip coordinates are automatically registered to the TRUS probe, and may be plotted on the TRUS image. Initially, the needle may not reside in the ultrasound imaging plane and may therefore not appear on the ultrasound image. Showing the location of the needle tip on the ultrasound image allows the position and/or orientation of the TRUS probe to be adjusted so that the needle coil location, as plotted on the ultrasound image, aligns with the TRUS transverse imaging plane. The needle tip is then visible on the TRUS image. In the embodiment of FIG. 9 the sensor coils are shown integrated into a “gps” sleeve 920 that slides over a TRUS probe, to enable the needle tracking functionality with standard or traditional TRUS probes. The sleeve can include openings or windows 922 designed and configured to accommodate the transducer arrays of the TRUS probe. In other embodiments, the sensor array is integrated within a TRUS probe.

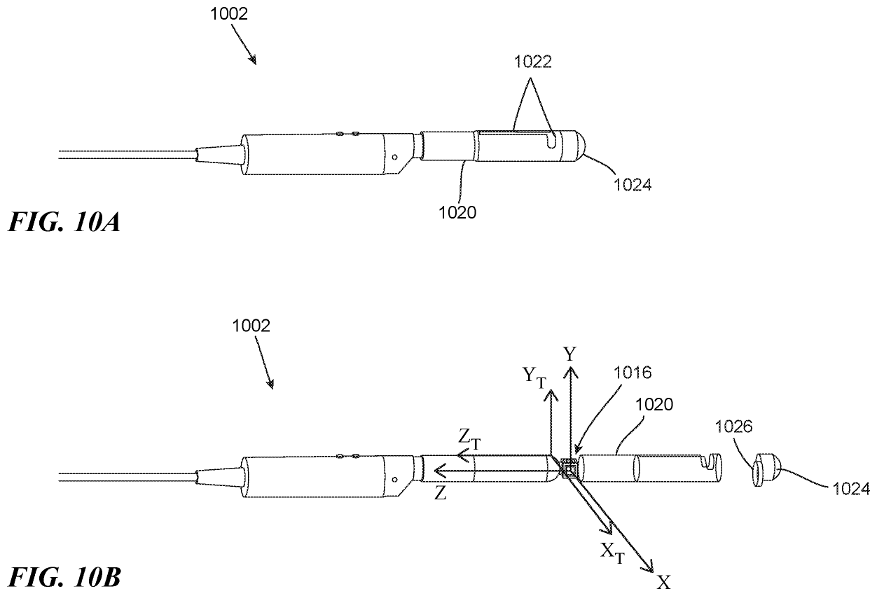

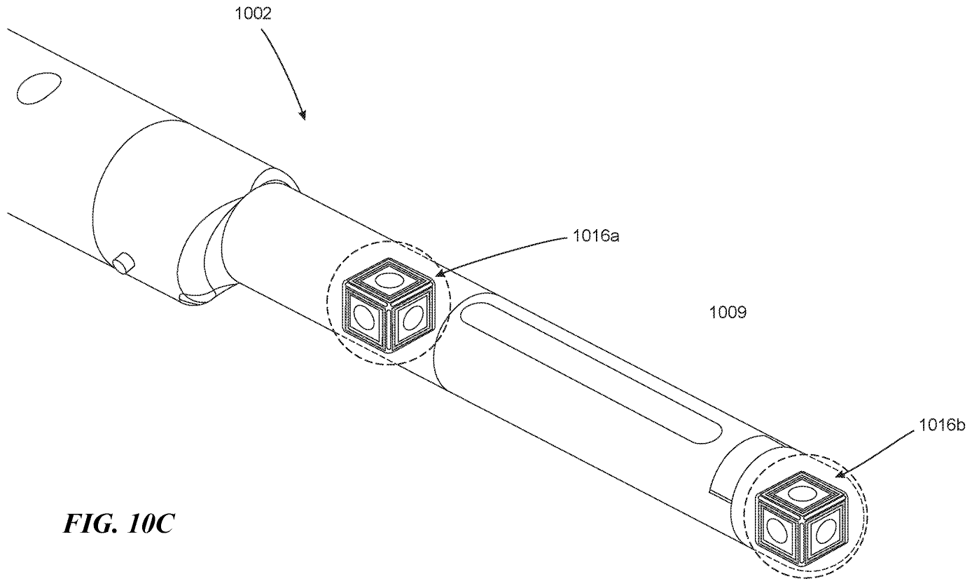

FIG. 10A shows a gps sleeve 1020 pushed or loaded onto a TRUS probe 1002, and FIG. 10B shows an exploded view of the underlying TRUS probe 1002, the gps sleeve 1020, the sleeve window 1022, and the sensor coils 1016. TRUS and gps coordinates are shown along the X, Y, and Z axis (gps coordinates) and X_{T}, Y_{T}, Z_{T} (TRUS coordinates) in FIG. 10B. The TRUS coordinate origin can be defined at the center of the central crystal of the transverse array of the TRUS probe. The gps coordinate origin can be defined at the center of the sensor coils 1016. As shown, the Y and Z gps coordinates are offset by fixed separations from the TRUS coordinates due to the axial separation between the TRUS transverse array and the sensor coils. When the gps coordinates of the needle tip are measured, the TRUS coordinates are given by a simple translation along the Y and Z axes. When the sleeve 1020 is loaded onto the TRUS probe, a nose cone 1024 of the sleeve can cover the sensor coils 1016 to protect the coils from bodily tissues and fluids while allowing for receipt of voltage signals from the needle sensor coil(s). The nose cone 1024 can be held in place on the sleeve with a locking ring 1026.

FIG. 10C shows an additional embodiment in which two arrays 1016a and 1016b of sensor coils are integrated into the TRUS probe 1002. In the illustrated embodiment, the two sensor cubes are placed in two locations proximal and distal to the TRUS transverse array 1009 as shown in FIG. 10C. Each array may have three orthogonal sensor coils, as shown, however it should be understood that the arrays can include more sensor coils. These may be individual coils wound around the cube circumferences or six coils wound on the cube faces with the outputs of the three pairs of coaxial coils summed to yield three orthogonal sensor outputs. The relatively large baseline between cubes allows for greater accuracy at all points in the space between the cubes. In addition, the formulas for the magnetic fields linking the sensor coils may be inverted analytically to provide theoretically exact solutions for the transmitter coordinates with no initialization requirements. In more general embodiments the sensor cubes may be rotated from each other by angles that optimize tracking accuracy.

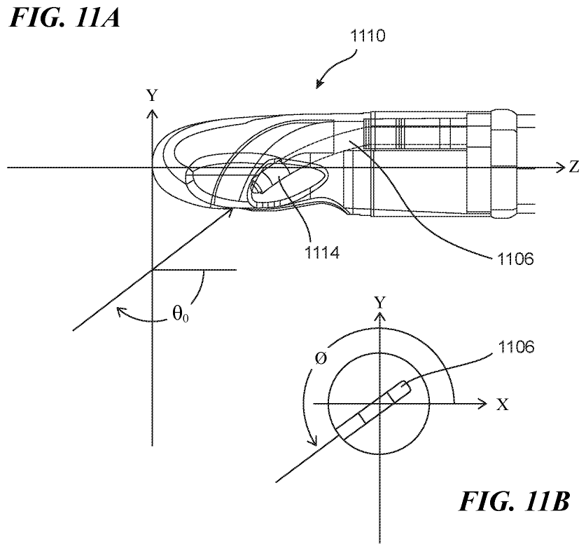

FIGS. 11A-11B show side and cross-sectional views, respectively, of the distal delivery device shaft tip 1110 and vapor delivery needle 1106 of this disclosure having a sensor or transmitter 1114. The polar and azimuth angles θ and φ are the same in gps sensor cube and TRUS coordinates because the gps sensor cube is rigidly mounted on the TRUS probe with X, Y, and Z axes parallel to each other. Initialization of the gps tracking system occurs at the start of a new procedure when the TRUS probe and the vapor delivery device shaft tip are at the base of the prostate. There are multiple indications of the location of the prostate base, for example: visually from the delivery device cystoscope; as seen on the TRUS sagittal image; and seeing the delivery device shaft tip in the TRUS transverse image. The gps Cartesian coordinates are estimated at this location with the needle exit port pointing down, (as seen in the TRUS or cystoscope images), and the vapor delivery device shaft parallel to the TRUS probe (as seen in the TRUS sagittal image). The X gps coordinate is equal to zero (same for TRUS and gps). The Y gps coordinate may be measured on the TRUS sagittal image as the separation between the TRUS and vapor probes plus the fixed separation between the centers of the central transverse crystal and center of the gps sensor cube. The Z coordinate is the known separation between the center of the gps sensor cube and the center of the central transverse TRUS crystal. The polar angle, θ, is equal to its initial value when the needle is retracted, θ₀, and the azimuth angle φ=270. With these initial estimates for the coordinates, the tracking software is started, and thereafter measures the coordinates and angles of the gps needle transmitter coil during the procedure. In one example, if the polar and azimuth angles are not equal to θ₀ and 270, the delivery device may be tilted until θ=θ₀ (delivery device shaft parallel to TRUS probe) and φ=270 (needle exit in the TRUS sagittal plane). In other embodiments initialization is not required because the equations for the sensed magnetic field may be inverted analytically to give the transmitter coordinates without need for an initial guess.

As the vapor delivery needle is deployed and advanced, the location of the transmit coil 1114 measured in gps coordinates is translated to TRUS coordinates and may be displayed on the TRUS images. The conversion is given by:

$${X}_{T} = {X}_{gps} \qquad ( 6 )$$

$${Y}_{T} = {Y}_{gps} - {Y}_{0}$$

$${Z}_{T} = {Z}_{gps} + {Z}_{0}$$

$${θ}_{T} = {θ}_{gps}$$

$${φ}_{T} = {φ}_{gps}$$

where
Y₀=vertical separation of cube center from center of central TRUS transverse crystal
Z₀=axial separation of cube center from center of central TRUS transverse crystal

Sensor guided needle targeting proceeds the same as Quantitative targeting described above except that movements of the vapor delivery device shaft and the shaft rotation angle φ are measured by the gps tracking system with the needle retracted. Once the needle is deployed, the actual location of the gps sensor coil is plotted on the TRUS images, along with the lesion centroid and other lesion details. Alternatively, the location of the vapor delivery holes proximate the gps coil, or the location of the needle tip may be plotted using their known translation from the gps coil. Vapor is delivered only when the vapor delivery holes are within a specified 3D separation from a target.

### GPS Tracking Systems and Methods

Commercial medical tracking systems typically include an array of transmit coils residing in a box placed outside the patient. Tens of Watts of transmit power are required to transmit measurable magnetic fields to the location of a catheter-mounted magnetic sensors within the patient, which may reside up to 50 cm from the transmit coil. The multiple transmit coils are energized sequentially so the catheter-mounted magnetic sensors can identify each individual transmit coil, or they transmit at distinct frequencies that are identified in sensor Fourier Transform software. One aspect of the present disclosure uses the needle tip coil as a transmitter (instead of a receiver of fields generated by multiple transmit coils as in traditional systems). With this design, the sensor coils can receive data simultaneously and continuously, reducing the time for noise to enter the system, or in the case of multiple frequencies, reducing the signal bandwidth and thereby reducing noise. Thus, the signal to noise ratio is increased in this disclosure.

Eddy currents induced in metal objects near the patient generate magnetic fields that are detected by sense coils. It is generally difficult to compensate for eddy current sources that arise from objects of arbitrary shapes and unknown locations. In the prostate cancer application, the transmit coil and sensor coils of this disclosure are typically separated by less than 10 cm during the procedure. The needle tip transmitted power can therefore be less than 0.1 Watt, compared to commercial transmit coils operating in the tens of Watts. The relatively very small transmit power from the needle tip coil will induce relatively small eddy currents in metal objects near the patient, resulting in relatively much smaller interfering magnetic fields. In the prostate application, the desired overall tracking volume is smaller than 200 cubic cm, residing entirely within the patient. It is very unlikely that foreign metal objects will reside within this volume. By contrast, the roughly 125,000 cubic cm tracking volume of commercial systems, with transmitters placed outside the patient, will very likely experience metal object interference.

Since the receive coils of this invention are mounted on the TRUS probe, needle tip tracking data is automatically given in TRUS image coordinates. Conventional systems, on the other hand, require that sensors be placed both on the needle and/or delivery device shaft, and on the TRUS probe, so that needle tracking data can be presented relative to the TRUS image.

This disclosure provides distinct ease of use advantages. The set of receive coils in this disclosure is integrated within the TRUS probe and the sensor cable is therefore integrated with the TRUS cable. The needle tip transmit coil leads are integrated into the cable extending from the delivery device cartridge to the system console. By contrast, commercial systems have one or more transmitter boxes that need to be set up, plugged into the system console or a separate box, and adjusted by the user. In many applications, leads extending from one or multiple sensors need to be plugged into the system console or a separate box.

### Tracking Systems and Methods

In a preferred embodiment, a set of theoretically exact equations is derived for the voltage induced in the six rectangular sensor coils by AC current flowing in the needle tip transmit coil. Rectangular sense coils are selected because highly accurate analytic expressions for the sensed voltages are available. Formulas for the voltages induced in the six rectangular coils located on the faces of a parallelogram by the magnetic field in the space around the needle tip transmit coil are expressed in terms of a Cartesian (x, y, z) coordinate system centered at the center of a parallelogram. As voltage data is collected, it is fit to the formulas by adjusting the assumed location and orientation of the transmit coil to find the least squares fit to the data.

In one preferred embodiment, as described above, the sensor cube (or other geometrical arrangement of sensors) is integrated into the manufacture of a TRUS ultrasound probe. The sensor coils are arranged for optimal tracking accuracy while never crossing the face of an ultrasound crystal. Sensor leads can be integrated with TRUS crystal leads into a single cable during manufacture. The sensor coils may be wound on a cube, as shown in FIGS. 9 and 10, or they may be integrated into the walls or body of the TRUS probe. Theoretically five sensor coils are needed to resolve the three Cartesian coordinates and two orientation angles of the transmit coil. In practice, better resolution is achieved when six or more coils are used.

### Shaft Mounted Transmitter

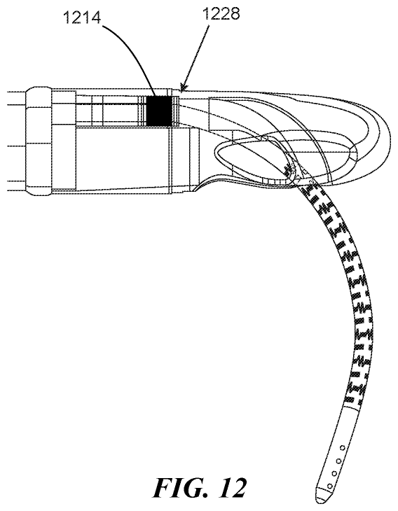

In some embodiments of this disclosure, a gps transmitter coil 1214 may be located on or in the delivery device shaft, as shown in FIG. 12 with the transmit coil wound on an exit tube or lumen 1228 within which the vapor delivery needle is slidably disposed. The location of the needle tip is estimated relative to the transmit coil location using the needle arc equation discussed above. The orientation of the plane of the needle can be determined in this embodiment by a rotation sensor (not shown) that is in communication with the vapor delivery needle at some location within the device shaft. The shaft mounted coil 1214 can be larger than the needle tip mounted coil (described above) to provide a stronger transmitted magnetic field and therefore a more accurate measurement. The shaft mounted coil can use larger diameter wire and be easier to construct and more cost effective. In other embodiments, a voltage induced into a needle tip coil or shaft mounted coil by a set of transmit coils located proximate the TRUS probe can be analyzed in real time to provide needle tip tracking.

### Sensor Electronics and Analog Signal Processing

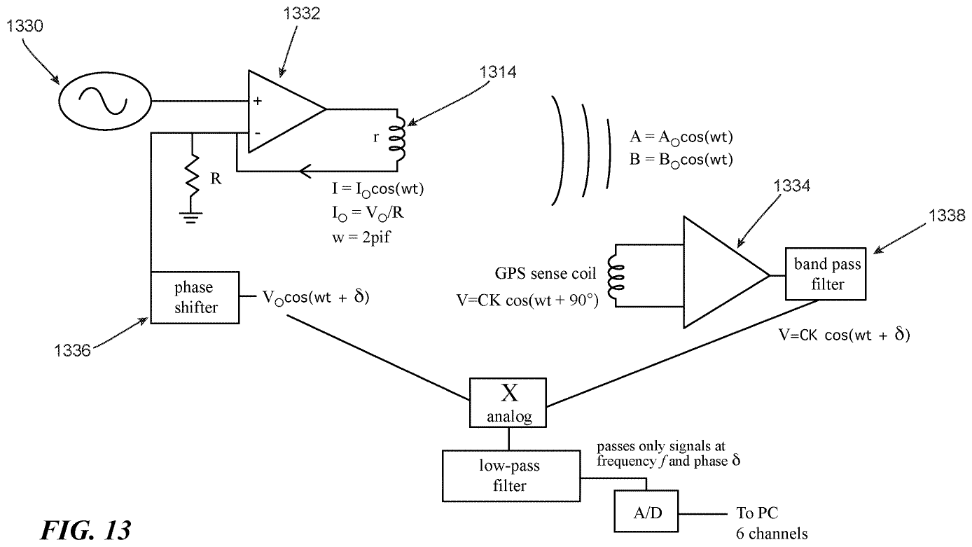

A preferred embodiment of drive coil electronics and sensor signal processing electronics of this disclosure is shown in FIG. 13. A precision AC sinusoidal waveform generator 1330 provides a stable input to the needle tip coil 1314 (or to transmit coils located proximate the TRUS probe when the needle tip coil is a sensor). The input voltage is defined as V₀ cos(ωt), where V₀ is the precision voltage amplitude and the angular frequency ω=2πf, where f is the drive frequency in Hz. The sinusoidal voltage is converted to current through the needle tip transmit coil by an operational amplifier 1332 with the coil in the feedback loop as shown. The precision current amplitude I₀ is equal to the ratio of the precision voltage V₀ to a precision resistor R. The power dissipated in the needle tip coil (=I₀²r/2, where r is the resistance of the needle tip coil) must be less than a maximum power that increases the coil temperature to a maximum safe value for human tissue contact. In one example the maximum safe coil power is 0.1 Watts. In another example the maximum safe power is 0.075 Watts. In one example the needle tip coil resistance at body temperature is 46 Ohms, and the maximum safe current is between 0.040 and 0.050 amps rms.

FIG. 13 also provides a schematic of analog signal processing electronics for one TRUS probe coil sensors. The transmit current and vector potential and magnetic field are at frequency f as shown. The transmit current is measured and is converted to a voltage with an amplifier 1334 that is phase shifted with phase shifter 1336 to be in phase with the sensed signals. Multiplying the phase shifted transmit voltage by the sensed voltage and averaging or low pass filtering with a band pass filter 1338 at a low frequency (e.g. 16 Hz) rejects any sensed voltages that are not at frequency f and phase δ.

In some examples, the needle tip coil is a magnetic dipole transmitter, creating a magnetic dipole vector potential A=A₀ cos(ωt) and magnetic field B that are known functions of the vector location of the needle tip relative to the TRUS probe sensors. The voltages induced in the six probe sensor coils are equal to minus the time rate of change of the magnetic flux linking each coil. The induced voltage is therefore proportional to sin(ωt)=cos(ωt+90°), that is, the induced voltages are 90 degrees out of phase with the drive current, as shown in FIG. 13. The sensed voltages are amplified and then band pass filtered. The filter introduces an additional phase shift. The net phase shift of the voltages relative to the current is defined as δ. The analog signal processor is comprised of an analog multiplier that multiplies the sensed voltage by a phase shifted measurement of the drive current. The phase shift is set by simultaneously observing the amplified and filtered sensor voltage and the phase shifted current drive voltage on an oscilloscope. The current phase shift is adjusted until the phase shifted current and sensor voltage are in phase (or 1800 out of phase depending upon the relative sign of the current and voltage). Since the electronics are the same for all six sensors, the phase shift is approximately the same for all sensors. In alternative embodiments, a separate phase shifter may be applied for each of the six sensors. In other embodiments, the phase shift is adjusted electronically to ensure that the inputs to the multipliers are in phase with each other at all times.

While the power dissipated in the needle tip is independent of drive frequency, f, the sensed voltages are proportional to frequency. For this reason, higher frequencies are preferred. Conventional tracking systems generally operate at frequencies that are less than 4 kHz. One reason for using lower frequencies is to minimize the impact of eddy currents induced in nearby metal objects. The voltage induced in an induction sensor by eddy currents in metal objects is proportional to the square of frequency, so lower frequencies are preferred to reduce this noise relative to internal sensor noise. Since the sensor voltage increases with frequency, a preferred operating frequency optimizes the signal to noise ratio. The proximity of the needle transmitter coil to the sensor coils in this invention minimizes the influence of eddy currents. Therefore, operating frequencies greater than 4 kHz may be used in this invention to increase the signal to noise ratio. In some embodiments the preferred operating frequency is between 4 and 10 kHz. In other embodiments the preferred operating frequency is >10 kHz.

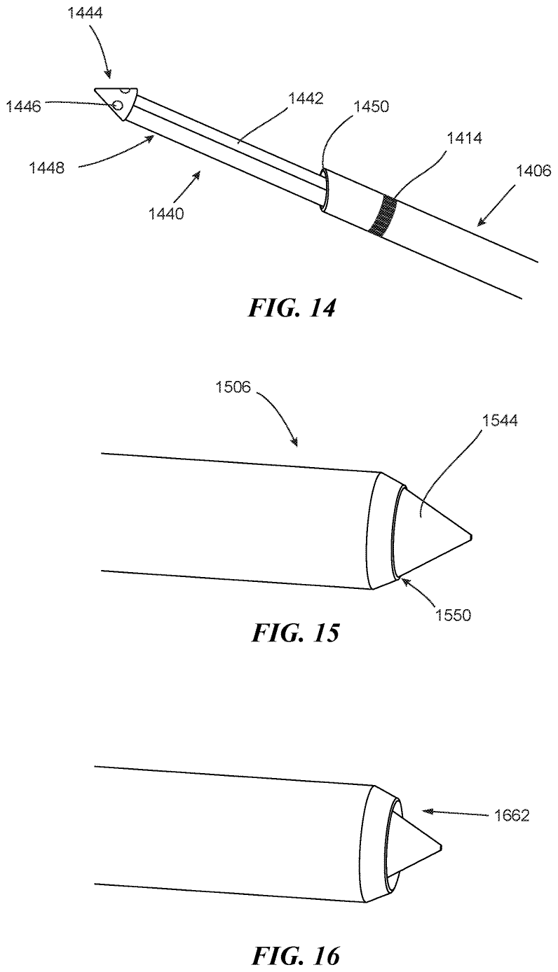

Analog signal processing, as shown in FIG. 13, is preferred in this disclosure to further optimize signal to noise ratio. In digital systems the sensor signal is amplified, low pass filtered, and digitized. Signal processing is performed in software. The digitizer or A/D converter has a limited accuracy and limited digitization rate. Generally, higher accuracy (more digital bits) requires more time per sample, or lower conversion rate. An accurate digital representation of the sensor sine wave signal requires a minimum number of bits per cycle of the sine wave. As operating frequency is increased, higher conversion rates are therefore desired. Higher conversion rates may require a reduction in number of bits of accuracy, thereby effectively limiting the digital signal to noise ratio. This issue is mitigated in the analog signal processor comprising the phase shifter and analog multiplier. Multiplication of the sine waves is continuous (not rate limited), and the DC product of the multiplication may be digitized at a low rate with many bits. Lower sampling rates reduce the bandwidth, thereby reducing the noise, while higher bit count samples allow accurate representation of the data down to and even below the noise level. In an example, 24 bit data is sampled at a conversion rate of 16 Hz (chosen to both give time to perform the calculations described above and be fast enough to appear like a continuous track on a video monitor). Since the analog signal processor of FIG. 14c filters the output at 16 Hz, the 24 bit A/D converter can digitize at any rate higher than 16 Hz without increasing the signal to noise ratio. Example digitization rates are 100 Hz or 1,000 Hz. Analog Devices Inc. AD7124 24 bit A/D converter has a sampling rate of 19.2 kHz. Data can be sub-sampled or averaged to the 16 Hz system display rate.

### Mutual Inductances

Currents are induced in the six coils of the sensor cube that are limited by the impedance of the sensor coils. These currents are small, but they do couple to neighboring coils through the mutual inductances between coils. However, the voltages induced by the mutual inductances are 90 degrees out of phase with voltages induced by the externally applied AC magnetic field and are therefore eliminated in the analog multiplier filter stage of the electronics.

It may be appreciated that many other medical device procedures may benefit from the miniaturized tracking system of this invention.

### Transurethral Biopsy Methods

In an embodiment of this disclosure, transurethral biopsy is performed before vapor therapy as a preferred way to acquire prostate tissue samples in desired locations, and particularly in tissues proximate those identified as potentially cancerous on a pre-operative MRI. The TRUS guided methods of lesion targeting of this disclosure apply equally to transurethral vapor therapy and transurethral biopsy. Biopsies may be directed to targeted tissues and may also be spaced throughout the prostate gland at the locations of a conventional 12 core transrectal biopsy. Biopsy samples may be acquired from sites that are difficult or impossible to reach with conventional transrectal biopsy, for example in and around the prostate apex and in anterior tissues.

Transurethral biopsy samples enter and leave the prostate into the urethra, an external body space. Any debris left in the urethra will be flushed out by sterile urine. By contrast, transrectal and trans-perineal biopsies enter and exit prostate tissue through intervening tissue. Cells may be carried from the intervening tissue into the prostate, and especially in transrectal biopsy, may include bacteria that could potentially cause sepsis. Cells from the prostate may be left in the intervening tissue upon exit, especially concerning if these are prostate cancer cells that may metastasize.

The transurethral biopsy system of this disclosure may be identical to or share the same form factor and system components as the vapor delivery systems described above, including system console, sensors and tracking devices, TRUS images with animations, pre-operative MRI scans, cystoscope images, therapy device stabilization system, methods of use, etc. While pre-therapy biopsy tissue samples may guide targeted vapor therapy, post-therapy biopsy samples may be used to assess the efficacy of tissue ablation.

Application of vapor as a biopsy needle is extracted from the prostate may cauterize the entrance hole in the urethra and prevent bleeding. Alternatively, a short burst of current applied between electrodes adjacent to the needle tip as it exits the prostate may cauterize blood vessels in tissue around the exit hole. The tip electrodes may provide bio-impedance (biocap) data throughout the procedure by measuring the voltage between them when a small AC current is passed through the adjacent tissues.

### Transurethral Biopsy Systems

In a preferred embodiment of this disclosure a biopsy needle that contains a tissue sample collector is deployed into the prostate through the wall of the urethra as previously described for vapor therapy. The needle may be similar in dimensions and materials to a vapor therapy needle, and can be deployed in a similar manner to how the vapor delivery needle is deployed above and herein. In one aspect, the biopsy needle can be advanced to the most proximal location of a desired biopsy tissue sample. A sample collector of the needle can then be advanced from the needle tip to a location that represents the most distal location of the desired biopsy tissue sample. The needle is then rapidly deployed from its proximal location to the location of the tip of the sample collector. The needle tip has a cutting edge that cuts the tissue and packs it into the needle along the length of the sample collector.

FIG. 14 shows one embodiment of a biopsy collection system including a biopsy needle 1406. This embodiment is similar to the vapor delivery system configured for transurethral access to the prostate (e.g., the biopsy needle can be delivered into prostate tissue transurethrally from an elongate device shaft as depicted in FIGS. 2, 8, 9, 11A, and 12 and can include a sensor 1414. However, the biopsy needle 1406 can be used in place of the vapor needle previously described. In some aspects, the vapor delivery needle of the vapor delivery needle can be swapped out for the biopsy needle of FIG. 14. In other aspects, a system is provided in which the needle is capable and configured to capture biopsy samples and also to deliver vapor into the prostate.

As shown in FIG. 14, the biopsy needle 1406 can include an extendable and retractable sample collector 1440 which extends out of the needle 1406. The sample collector can include a sample trough 1442 for collecting biopsy tissue samples. In some aspects, the sample collector includes a tip 1444 which may include one or more electrodes 1446. The electrodes can be provided for sensing (e.g., bioimpedance sensing) or for applying current to tissue (e.g., cauterization). The electrodes can be used to sense impedance in the tissue for placement near the biopsy tissue to be collected. The electrodes can also be used to cauterize the tissue when the needle is removed, after the biopsy tissue sample has been collected. Leads 1448 can be integrated into the sample collector to provide current to the electrodes 1446. When the sample collector 1440 is deployed or advanced out of the needle 1406, it can expose a cutting edge 1450 of the needle. As with other embodiments described herein, the needle itself can include sensors or transmitters configured for tracking needle position.

The biopsy needle and sample collector tip are shown in their nominal configuration in FIG. 15. The needle and tip move together during needle deployment through the urethra and into the prostate, and during needle retraction into the delivery device shaft.

In some embodiments, the sample collector and needle are removed together along with the needle control mechanism. Once outside the body, the sample may be removed by extracting the sample collector from the needle as shown in FIG. 16, removing the sample, and replacing the sample collector and needle into the delivery device. In other embodiments the needle may remain in the body, for example in the prostate or in the delivery device shaft, while the sample collector is removed from the needle as shown in FIG. 16. The sample collector may then be replaced by a new sample collector, or the sample may be removed, and the sample collector re-delivered to the needle tip.

In some embodiments the needle tip may be deflectable so that the sample collector may take a new path through tissue with a single puncture of the urethra wall. When the needle and sample holder are retracted into the delivery device shaft, the shaft may be moved to a new location and orientation in the urethra using the targeting methods of this invention, and then deployed through the urethra wall to obtain a tissue sample in a new location.

Samples may be obtained in tissue suspected of being cancerous in an MRI image of the prostate, or samples may be taken in random locations throughout the prostate in analogy with a standard 12 core biopsy.

To perform a biopsy procedure, the needle 1506, with the sample collector retracted such that only the tip 1544 is exposed against the cutting edge 1550 of the needle, as shown in FIG. 15, is deployed through the wall of the urethra at a site determined by the targeting methods of this disclosure. The needle is advanced to a location that will be the proximal end of the tissue sample. At this point, the sample collector is extended with the needle held rigidly in place (as shown in FIG. 14), to a location that will be the distal end of the sample. With the sample collector and distal tip held rigidly in place, the needle can then be advanced rapidly towards the tip to cut the tissue with the cutting edge and capture it within the trough 1442 of the sample collector. Once the needle is advanced to the tip of the sample collector, the entire biopsy needle, including the collected tissue sample may be removed. Removal can be accomplished by retracting the sample collector, as shown by arrow 1662 in FIG. 16, proximally through the needle and out of the needle and/or the device. The needle may be retracted into the urethra along with the sample collector before the sample is removed. In some embodiments, the needle has an inside diameter in the range of 1.0 mm to 1.5 mm, and the length of the sample collected in the sample collector trough is in the range of 5 mm to 15 mm.

### Auger Biopsy Tool

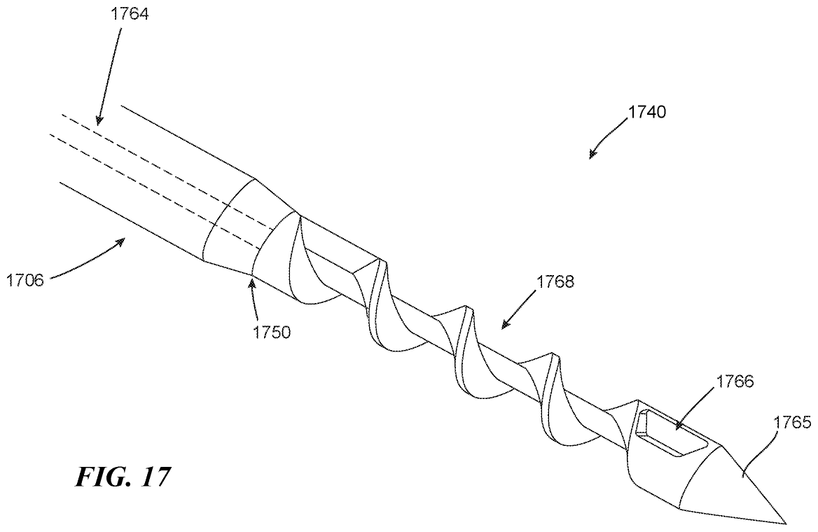

FIG. 17 shows one example of a biopsy needle 1706 that includes a sample collector 1740 with a different design than is described above. In this example, the sample collector can comprise an auger biopsy tool that can be attached to a rotatable cable or shaft 1764 that extends through the needle to the proximal end of the biopsy system. Rotation of the shaft or cable causes rotation of the auger sample collector 1740, for example with a screw or motor in the device that has the same pitch as the auger. As the auger rotates, the auger tip 1765 and grooves 1768 extend out of the needle and into tissue. The auger can include a cutting window 1766 that slices the tissue that then moves proximally into the grooves 1768 of the auger. Finally, the needle 1706 including the needle cutting edge 1750 that can be advanced rapidly to cover the auger sample collector and cut off any remaining tissue in the cutting window. The biopsy needle can then be removed as previously described above.

In one preferred embodiment the outside diameter of the biopsy needle is 1.25 mm-1.5 mm and the outside diameter of the auger cutting edges is 1 mm-1.25 mm. The separation between auger blades is 1.25 mm, and the auger advances by 1.25 mm per revolution. A sample length of 5 mm is obtained by rotating a four-blade auger through four revolutions. A sample length of 10 mm is obtained by rotating an eight-blade auger through eight revolutions.

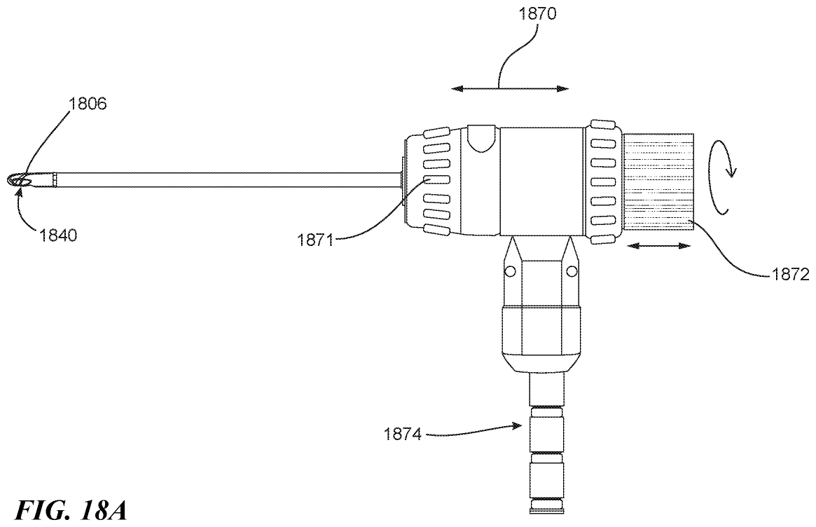

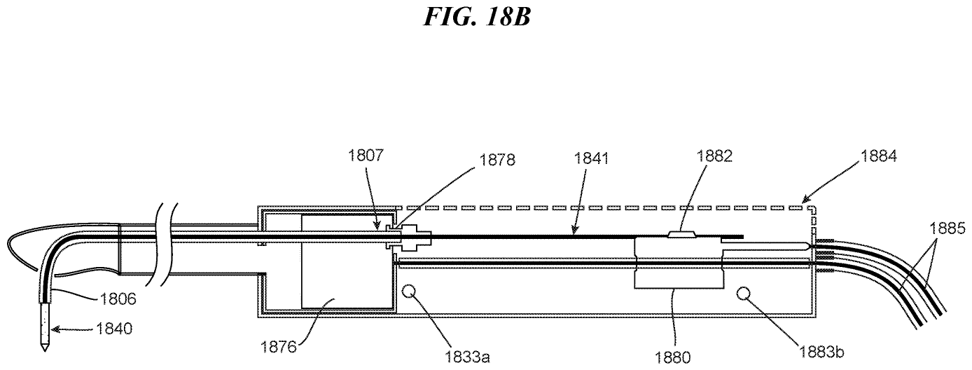

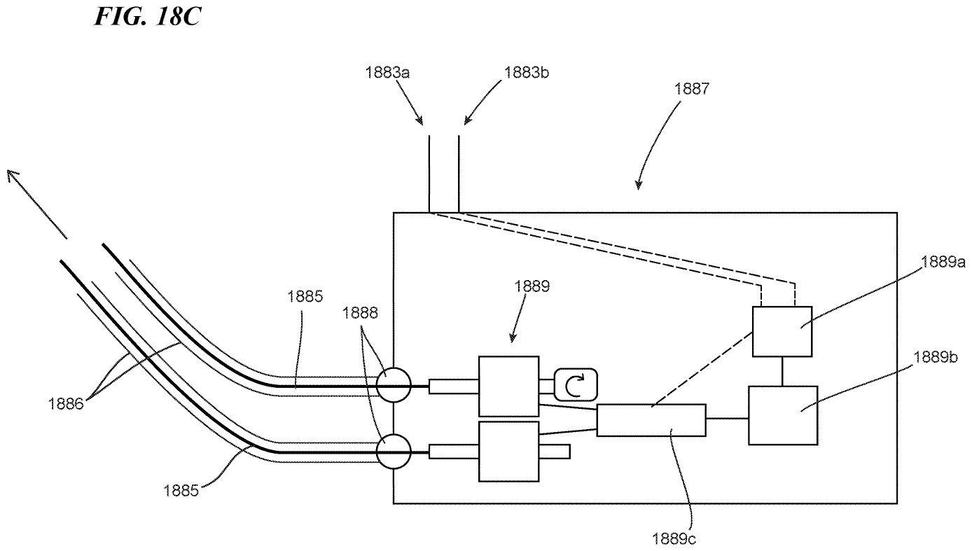

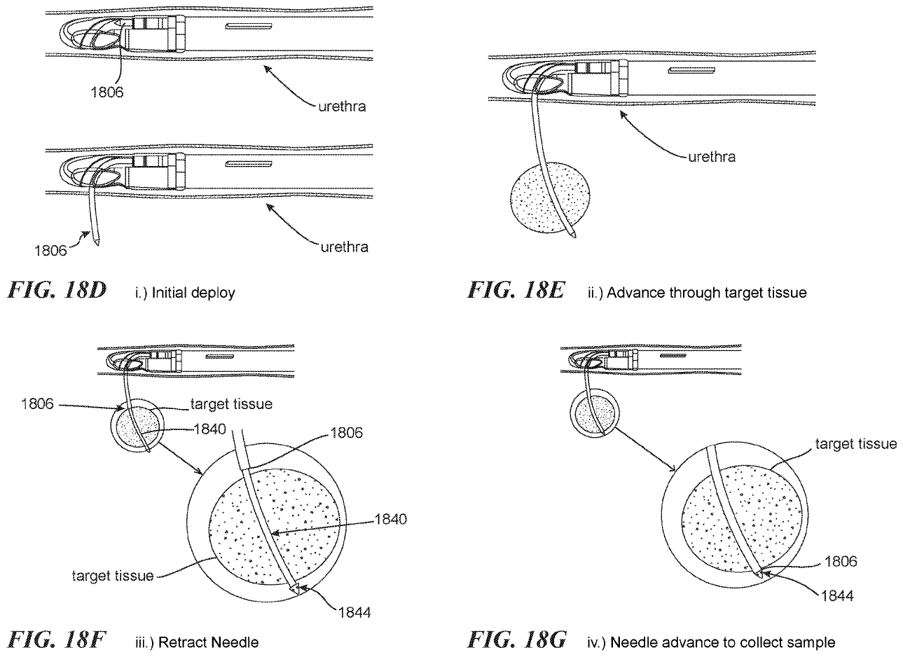

A prostate biopsy delivery device of this disclosure is shown in FIG. 18A. A sample collector 1840 is shown advancing from the biopsy needle 1806. The shaft is inserted into the urethra guided by a cystoscope video image and TRUS images. Tissue to be extracted is identified and targeted using the methods of this patent. The biopsy needle is controlled by an advance/retract mechanism 1870 within the device which may be a push-pull solenoid, linear motor, hydraulic actuator, spring mechanism or other actuation mechanism. The needle may also be rotated with rotation knob 1871. The sample collector is shown in FIG. 18 being controlled by a separate advance/retract mechanism 1872 which may also be rotated in embodiments with the auger tissue collector. The entire device can be supported with a support assembly or arm 1874, as shown.

During needle deployment to target tissue through the wall of the urethra, the needle and sample collector are advanced together in the configuration of FIG. 15.

In some embodiments, referring to FIGS. 18B-18C, the needle 1806 and sample collector 1840 may be connected via flexible cables to linear motor drivers located in the system console. In FIG. 18B, a proximal portion 1807 of the needle 1806 is removably connected to a needle carriage 1876 with a removable needle attachment 1878, and a shaft 1841 of the sample collector 1840 is removably connected to a sample collector carriage 1880, such as with a clamp 1882 or other attachment means. Needle position sensor 1883a and sample collector position sensor 1883b can track the relative positions or deployment distances of the needle and sample collector, respectively. A cover 1884 may be closed over the delivery device cartridge when it is in use. Flexible drive shafts 1885 can be attached to linear motors, described below in FIG. 18C.

Referring to FIG. 18C, the drive shafts 1885 from the needle and sample collector carriages can be housed in flexible compliant sheaths 1886 which lead into the linear motors 1889 of the system console 1887 via quick connects 1888. The console can include electronics 1889a-c which may include one or more processors/cpus, a power supply, and a controller configured to control the linear motors to manage needle and sample collector position, such as based on sensor inputs from the needle and sample collector sensors 1883a and 1883b.

This system enables all sample collection options described herein via independent control of the sample collector and needle. Locating the driver(s) in the console reduces the size and weight of the biopsy device cartridge. Commercial linear motors may supply pounds of holding force at any location with up to tens of pounds of thrust force and speeds up to 7 m/sec with programmable control of all needle movements. The linear motors are small enough to fit into the system console. The drive cable may connect to the console via a quick connect mechanism. The motor shaft may be rotated by an electric motor for auger applications. Remote drive linear motors and actuators may be employed in any device described herein.

In another tissue biopsy collection method as shown in FIGS. 18D-18G, the device shaft is positioned within the urethra of a patient with the target tissue positioned radially or to the side of the device shaft. During the initial deployment shown in FIG. 18D, the needle 1806 and sample collector are deployed from within the device shaft to be out of the shaft and extend through the urethra into the prostate tissue. In FIG. 18E, the needle and sample collector are then advanced through the target tissue until the needle tip is distal of the most distal end of the targeted tissue sample. At this point, referring to FIG. 18F the sample collector tip 1844 is held in place while the needle 1806 is retracted through the targeted tissue until its needle tip is beyond the most proximal end of the tissue sample. As the needle is retracted, tissue is urged into the sample collector 1840 due to tissue elasticity and the arc of the sample collector. Referring to FIG. 18G, the needle can then be rapidly advanced over the sample collector 1840. The needle cutting edge as described above can also be configured to sever tissue while its beveled shape pushes the severed sample into the sample collector 1840. The needle and sample collector can then be retracted into the shaft and removed from the delivery device. The sample collector may be pushed forward to expose the sample and remove it to a collection pad for histological examination. The sample collector and needle are cleaned, for example by movement through a sanitizing fluid, and reinserted into the delivery device to collect the next sample.

### Mechanical Auger Biopsy Systems and Methods

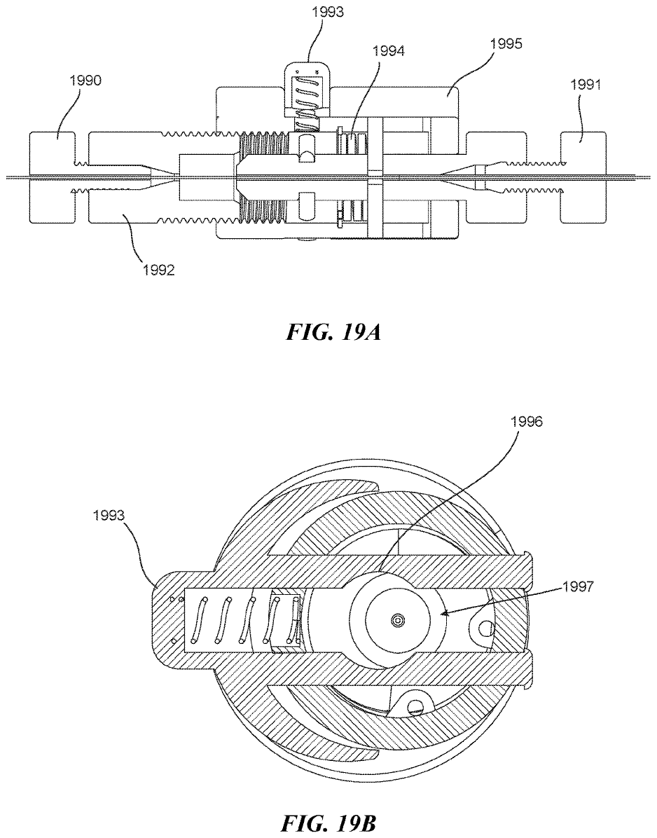

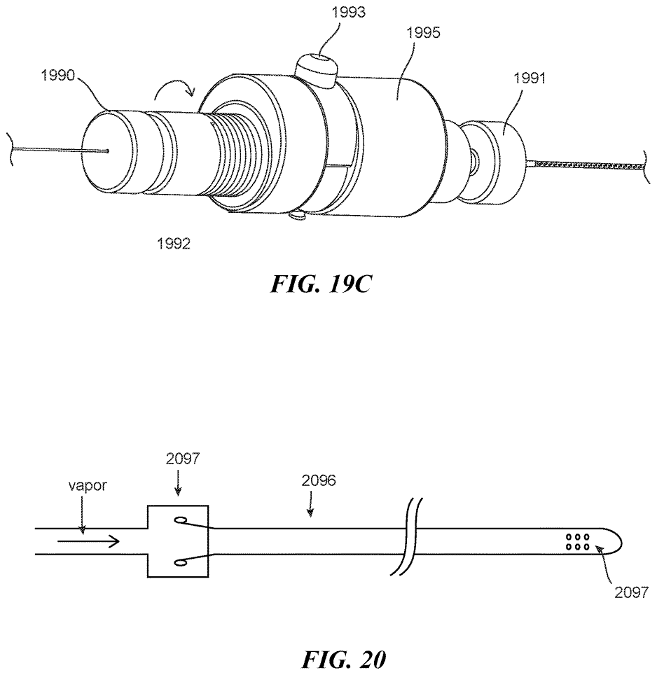

One embodiment of a purely mechanical mechanism to rotate and advance an auger sample collector of this invention, followed by advancement of the needle, is shown in FIGS. 19A-19C. FIG. 19A shows one embodiment of a mechanical auger tissue sample collector of this disclosure, which can be used with the auger biopsy tools described herein and above. In this example, sample collet 1990 grips the auger and needle collet 1991 grips the needle, which enables them to move separately or together. The auger is advanced and simultaneously rotated by turning the auger rotation knob 1992. The needle advances via needle advance button 1993 through threads that have the same pitch as the auger shown above. The auger advances in each 360° rotation by a distance equal to the separation between auger blades. Once the auger has been fully advanced, the needle with cutting edge is propelled rapidly by the auger advance spring 1994 (which is released by pushing the needle advance button 1993) to cover the length of the tissue sample and cut off tissue in the cutting window. This biopsy tool may be used separately or in combination with a delivery device by mounting to the mounting surface 1995.

FIG. 19B shows another view of the needle advance button 1993, which can be a spring loaded needle advance mechanism. When the needle advance button 1993 is pushed, a spring release window 1996 moves to allow the needle advance spring 1997 to release, advancing the needle by the fixed length of the auger.

FIG. 19C provides an external view of an auger tissue sample collector mechanism, including the auger collet 1990, the auger rotation knob 1992, the needle collet 1991, the needle advance button 1993, and the mounting surface 1995.

The auger is clamped into the auger block by the sample collector collet threading into the auger block in FIG. 19A. The needle is clamped into the needle block by the needle collet threading into the needle block. The entire assembly is advanced into position. A linear control system, for example the push-pull controller in the vapor therapy cartridge, may be attached to the mounting surface seen in FIGS. 19A and 19C to advance/retract the entire mechanism of FIG. 19A. To advance the auger into the target tissue, the auger block is threaded into the main assembly with the same pitch as the auger. The auger advances 10 mm in this example. The needle is “snapped” over the auger by the needle block spring that is released when the needle advance button (with an internal return spring) is pressed, as shown in FIG. 19B. The needle block spring propels the needle block distally until the pins bottom out in the 10 mm long slots in the main assembly. The needle is then at its most distal location with the sample collected within.

The sample collector collet can be loosened to permit the auger to be removed from the assembly, or both the sample collector and needle collets can be loosened to permit the removal of the auger and needle as a unit. The needle is manually fed through the assembly until it can be grasped at the proximal end.

The button is held open by the proximal stem in the needle block until the needle collet is manually pushed back to re-latch the needle block after reloading the auger and/or sheath.

### Vapor Delivery and Biopsy

FIG. 20 shows a vapor delivery needle liner 2096 which can be inserted into any of the biopsy needles described herein following tissue biopsy to cauterize the needle exit hole in the wall of the urethra as the needle is retracted into the delivery device shaft.

In an embodiment of this disclosure, the tissue sample collector is removed from the needle while the tip of the needle is in prostate tissue. The sample collector is replaced by the needle liner 2096 shown in FIG. 20. The needle liner is connected to a source of vapor at its proximal end, such as with one or more luer fittings 2097. The liner is advanced through the needle until the vapor delivery holes or ports 2098 extend from the needle tip into tissue (e.g., out of the biopsy needle shaft). In one embodiment, the needle and liner are retracted through the wall of the urethra together while vapor is released to cauterize the needle exit hole. Vapor may be delivered at a low rate while the liner is advanced and at a higher rate as the needle and liner are retracted through the urethra wall. Vapor is stopped when the needle is retracted into the delivery device shaft. In one embodiment, vapor exits the distal end of the needle (with sample collector removed), without using a liner. In some embodiments vapor therapy is performed followed by biopsy to assess tissue ablation. In other embodiments, vapor therapy is delivered at sites where biopsy tissue samples have been removed and found to be cancerous. Other combinations of biopsy and vapor therapy may be envisioned.

Although embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. Variations and alternatives will be apparent to one having ordinary skills in the art. Such alternatives and variations are intended to be included within the scope of the claims. Features that are presented in dependent claims can be combined and fall within the scope of the invention. The invention also encompasses embodiments as if dependent claims were alternatively written in a multiple dependent claim format with reference to other independent claims.

1. A method, comprising:
obtaining a first medical image of a prostate including target tissue region and an anatomical landmark;
measuring a separation distance and clock angle between the target tissue region and the anatomical landmark;
imaging the anatomical landmark under real-time ultrasound imaging;
advancing a needle position of a vapor delivery device transurethrally to the anatomical landmark under the real-time ultrasound imaging;
further advancing and rotating the needle position of the vapor delivery device transurethrally by the separation distance and clock angle; and
deploying a needle of the vapor delivery device from the needle position through the urethra, into the prostate, and into the target tissue region.

2. The method of claim 1, wherein the target tissue region comprises prostate cancer tissue.

3. The method of claim 1, wherein the anatomical landmark is selected from the group consisting of a base of the prostate, an apex of the prostate, a bladder neck, a verumontanum, and a location where ejaculatory ducts meet the urethra.

4. The method of claim 1, wherein the real-time ultrasound imaging comprises a trans-rectal ultrasound imaging (TRUS) probe.

5. The method of claim 4, wherein further advancing the needle position further includes advancing the TRUS probe in step with the needle position of the vapor delivery device.

6. The method of claim 1, wherein deploying the needle further comprises deploying the needle generally transverse to a shaft of the vapor delivery device.

7. The method of claim 1, wherein deploying the needle further comprises deploying the needle along an arc.

8. The method of claim 7, further comprising displaying an animation of the arc of the needle on the real-time ultrasound image, translating and/or rotating the delivery device until the animated needle tip intersects an animation of targeted cancerous tissue, then deploying the needle into the target tissue.

9. The method of claim 1, further comprising tracking a position of the needle during the deployment.

10. The method of claim 9, further comprising displaying the tracked position on the real-time ultrasound imaging.

11. A medical device gps tracking system, comprising:
a trans-rectal ultrasound imaging system (TRUS) probe configured to obtain one or more ultrasound images of a target tissue in an imaging plane;
a plurality of sensors disposed on or within the TRUS probe, the plurality of sensors being configured to receive signals from one or more transmitters positioned on a medical device and within the imaging field of view; and
control electronics configured to determine a position of the one or more transmitters from the received signals and communicate position data of the medical device for real time display on the one or more ultrasound images.

12. The system of claim 11, wherein the plurality of sensors are disposed on a sleeve configured to be placed over the TRUS probe.

13. The system of claim 12, wherein the TRUS probe comprises a transverse transducer array, wherein the sleeve has a window or opening corresponding to the transverse transducer array.

14. The system of claim 11, wherein the plurality of sensors on or within the TRUS probe comprise induction sensor coils.

15. The system of claim 14, wherein the induction sensor coils comprise six induction sensor coils.

16. The system of claim 14, wherein the induction coils are wound on faces of a non-metal sensor cube.

17. The system of claim 14, wherein the induction coils are positioned on a distal tip of the TRUS probe.

18. The system of claim 14, wherein the induction coils are positioned distal and proximal to a sagittal array of the TRUS probe.

19. The system of claim 14, wherein the control system is configured to register coordinates of the TRUS probe to coordinates of the plurality of sensors.

20. A medical system, comprising:
a therapy device configured for transurethral access to a patient's prostate, the therapy device having a deployable needle configured to be actuated from a delivery configuration to a deployed configuration and one or more transmitters disposed thereon;
a trans-rectal ultrasound imaging system (TRUS) probe;
a plurality of sensors disposed on or within the TRUS probe, the plurality of sensors being configured to receive signals from the one or more transmitters;
control electronics configured to determine a position the one or more transmitters from the received signals and communicate position data to the TRUS probe; and
a display configured to display real-time images of the prostate from the TRUS probe overlaid with the position of the one or more transmitters.

21.-43. (canceled)
